(19) United States
(12) Patent Application Publication
(10) Pub. No.: US 2017/0281264 A1
(43) Pub. Date: Oct. 5, 2017

(54) ELECTROSURGICAL DEVICE FOR VESSEL SEALING

(71) Applicant: Gyrus ACMI, Inc., d.b.a. Olympus Surgical Technologies America, Southborough, MA (US)

(72) Inventors: Huisun Wang, Maple Grove, MN (US); Kester J. Batchelor, Mound, MN (US); Jeffrey Nelson, Maple Grove, MN (US)

(21) Appl. No.: 15/470,166

(22) Filed: Mar. 27, 2017

Related U.S. Application Data
(60) Provisional application No. 62/317,858, filed on Apr. 4, 2016.

(51) Int. Cl. A61B 18/14; A61B 17/29; A61B 17/08

(52) U.S. Cl. CPC A61B 18/1445; A61B 17/08; A61B 17/2909; A61B 2090/034

(57) ABSTRACT

An end effector assembly of a forceps includes a first jaw member having an electrically conductive tissue sealing surface configured to connect to a source of electrosurgical energy and a second jaw member having an electrically conductive tissue sealing surface configured to connect to the source of electrosurgical energy. The first and the second jaw members are disposed in space opposition relation relative to one another, and at least one of the jaw members is movable relative to the other between a first, open position and a second, closed position for the jaw members to grasp tissue therebetween. The tissue sealing surfaces of the first and the second jaw members are configured to form complementary stepped portions along an axis perpendicular to the longitudinal axis of the end effector assembly. The complementary stepped portions include a medial portion and a lateral portion on each of the first and second jaw, and one or both of the lateral surfaces has nonconductive stops.

### RELATED APPLICATION

The present application claims the benefit of U.S. Provisional Patent Application No. 62/317,858, filed on Apr. 4, 2016. The entire contents of the above application are incorporated herein by reference.

### FIELD

The present disclosure relates to an electrosurgical device. More specifically, the present disclosure relates to an electrosurgical device for vessel sealing.

### BACKGROUND

The statements in this section merely provide background information related to the present disclosure and may or may not constitute prior art.

Generally forceps may be utilized for laparoscopic surgery. The forceps may be employed to control delicate movements inside a patient and may include a gripping assembly or a cutting assembly. Further, the forceps may utilize electrical energy in the gripping assembly. Typically, the forceps have a pair of opposed resilient jaws that are closed against each other by pulling the jaws into a distal end of a shaft that captures a portion of the jaws that is wider than the distal end opening of the shaft so that the jaws are moved together. Similarly the shaft may be pushed over the jaws so that the jaws are moved together to create a gripping force. In both of these arrangements, the shaft captures the jaws and acts as a cam that forces the jaws together to create the gripping force. Examples of some forceps with resilient jaws closed by a camming action may be found in U.S. Pat. Nos. 5,458,598; 5,735,849; 5,445,638; 6,190,386; 6,113,596; and 6,679.882 and HALO cutting forceps, available at http://www.olympus-osta.com/halo.htm last accessed on Apr. 3, 2014, all of which are incorporated by reference herein in their entirety for all purposes.

Current bipolar electrosurgical sealing forceps employ a pair of jaws with RF energy to coagulate a vessel and further employ a moveable cutting blade to cut the sealed vessel after coagulation. Such devices, however, require a high jaw force to compress the vessel tissue for desired sealing results. The high jaw force can cause unwanted tissue damage and may also reduce the device durability and reliability.

Accordingly, it would be attractive for the electrosurgical forceps to not require high jaw forces for vessel sealing.

### SUMMARY

The present disclosure provides an electrosurgical bipolar forceps which does not require high jaw force for vessel sealing.

Accordingly, pursuant to one aspect, an end effector assembly of a forceps includes a first jaw member having an electrically conductive tissue sealing surface configured to connect to a source of electrosurgical energy and a second jaw member having an electrically conductive tissue sealing surface configured to connect to the source of electrosurgical energy. The first and the second jaw members are disposed in space opposition relation relative to one another, and at least one of the jaw members is movable relative to the other between a first, open position and a second, closed position for the jaw members to grasp tissue therebetween. The tissue sealing surfaces of the first and the second jaw members are configured to form complementary stepped portions along an axis perpendicular to the longitudinal axis of the end effector assembly. The complementary stepped portions include a medial portion and a lateral portion on each of the first and second jaw, and one or both of the lateral surfaces has nonconductive stops.

This aspect may be further characterized by one or any combination of the features described herein, such as: the sealing surface of the first jaw member includes a first compression surface along the medial portion, a second compression surface along the lateral portion, and a shearing surface between the first compression surface and the second compression surface; the sealing surface of the second jaw member includes a first compression surface along the medial portion, a second compression surface along the lateral portion, and a shearing surface between the first compression surface and the second compression surface; the shearing surface of each of the jaw members is arranged orthogonally to the first compression surface and the second compression surface of the respective jaw member; the shearing surface of each of the jaw members is arranged non-orthogonally to the first compression surface and the second compression surface of the respective jaw member; the non-conductive stop is a gripping member positioned along the outermost compression surface of at least one of the jaw members, the non-conductive stop preventing inadvertent shorting between the jaw members; and the source generates electrosurgical energy to coagulate tissue grasped between the first jaw member and the second jaw member.

Accordingly, pursuant to another aspect, a forceps with an effector assembly a first jaw member having an electrically conductive tissue sealing surface configured to connect to a source of electrosurgical energy and a second jaw member having an electrically conductive tissue sealing surface configured to connect to the source of electrosurgical energy. The first and the second jaw members are disposed in space opposition relation relative to one another, and at least one of the jaw members movable relative to the other between a first, open position and a second, closed position for the jaw members to grasp tissue therebetween. The tissue sealing surfaces of the first and the second jaw members are configured to form complementary stepped portions along an axis perpendicular to the longitudinal axis of the end effector assembly, the complementary stepped portions comprising a medial portion and a lateral portion on each of the first and second jaw. One or both of the lateral surfaces has nonconductive stops.

This aspect may be further characterized by one or any combination of the features described herein, such as: the sealing surface of the first jaw member includes a first compression surface along the medial portion, a second compression surface along the lateral portion, and a shearing surface between the first compression surface and the second compression surface; the sealing surface of the second jaw member includes a first compression surface along the medial portion, a second compression surface along the lateral portion, and a shearing surface between the first compression surface and the second compression surface; the shearing surface of each of the jaw members is arranged orthogonally to the first compression surface and the second compression surface of the respective jaw member; the shearing surface of each of the jaw members is arranged non-orthogonally to the first compression surface and the second compression surface of the respective jaw member; the non-conductive stop is a gripping member positioned along the outermost compression surface of at least one of the jaw members, the non-conductive stop preventing inadvertent shorting between the jaw members; the source generates electrosurgical energy to coagulate tissue grasped between the first jaw member and the second jaw member; and the tissue is gripped to provide tension and the forceps includes a reciprocating blade that cuts the tissue.

Accordingly, pursuant to yet another aspect, a method of using forceps includes one or more of the following steps: opening a first jaw member and a second jaw member of the forceps, the first jaw member having an electrically conductive tissue sealing surface configured to connect to a source of electrosurgical energy and the second jaw member having an electrically conductive tissue sealing surface configured to connect to the source of electrosurgical energy, the first and the second jaw members being disposed in space opposition relation relative to one another, the tissue sealing surfaces of the first and the second jaw members being configured to form complementary stepped portions along an axis perpendicular to the longitudinal axis of the end effector assembly, the complementary stepped portions comprising a medial portion and a lateral portion on each of the first and second jaw, one or both of the lateral surfaces having nonconductive stops; closing the jaw members to grasp tissue therebetween; and pressing the jaw members together to cut tissue.

The method of using the forceps may be further characterized by one or any combination of the following features: the sealing surface of the first jaw member includes a first compression surface along the medial portion, a second compression surface along the lateral portion, and a shearing surface between the first compression surface and the second compression surface; the sealing surface of the second jaw member includes a first compression surface along the medial portion, a second compression surface along the lateral portion, and a shearing surface between the first compression surface and the second compression surface; the shearing surface of each of the jaw members is arranged orthogonally to the first compression surface and the second compression surface of the respective jaw; the shearing surface of each of the jaw members is arranged non-orthogonally to the first compression surface and the second compression surface of the respective jaw member; the non-conductive stop is a gripping member positioned along the outermost compression surface of at least one of the jaw members, the non-conductive stop preventing inadvertent shorting between the jaw members; generating electrosurgical energy to coagulate tissue grasped between the first jaw member and the second jaw member; and the tissue is gripped to provide tension and the tissue is cut by a reciprocating blade.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the invention. In the drawings:

FIG. 1 illustrates an electrosurgical forceps in accordance with the principles of the present invention;

FIG. 2 an example of a set of jaws for the forceps shown in FIG. 1;

FIG. 3 illustrates an end of a tubular member and/or a camming shaft for the forceps;

FIG. 4 illustrates an end view of a tubular member and/or a camming shaft;

FIG. 5 illustrates a perspective view of a camming shaft;

FIG. 6 illustrates a perspective view of the forceps shown in transparent;

FIG. 7A illustrates a cross-sectional view of the jaws sealing a vessel;

FIG. 7B illustrates a close-up cross-sectional view of the jaws;

FIG. 8 illustrates a cross-sectional view of an alternative embodiment of a set of jaws sealing a vessel in accordance with the principles of the present invention;

FIG. 9 illustrates a cross-sectional view of yet another alternative embodiment of a set of jaws in accordance with the principles of the present invention;

FIG. 10 illustrates a cross-sectional view of yet another alternative embodiment of a set of jaws in accordance with the principles of the present invention;

FIG. 11 illustrates a cross-sectional view of yet another alternative embodiment of a set of jaws in accordance with the principles of the present invention;

FIG. 12 illustrates a perspective view of the jaws shown in FIG. 6 with a cutting blade; and

FIG. 13 illustrates a side view of the jaws shown in FIG. 6 with the cutting blade.

### DETAILED DESCRIPTION

The following description is merely exemplary in nature and is not intended to limit the present disclosure, application, or uses.

Referring now to the drawings, a forceps, such as, for example, a laparoscopic forceps, embodying the principles of the present invention is illustrated therein and designated at 2. The forceps 2 may function to grip an object. The forceps 2 may be used during surgery to grip a feature of interest including: a part of a body, an anatomical feature, tissue, veins, arteries, or a combination thereof. The forceps 2 may function to be used in surgery, for example, laparoscopic surgery. The forceps 2 may be used with or without power. Current may be passed through the forceps 2 so that the forceps are used for electrosurgery. For example, a therapy current may be passed from one jaw to a second jaw when tissue is located within the jaw and the therapy current may coagulate blood, cauterize, cut, or a combination thereof. The forceps 2 may generally include one or more working assemblies and sufficient controls to work the one or more assemblies. The forceps 2 may include parts employed to perform the recited functions and may include generally, a stylet (e.g., a tubular member, a hollow tube, or an assembly of tubes), a hand piece, one or more operable mechanisms used to actuate the stylet, or a combination thereof. The hand piece may be an assembly of parts or housing structures capable of forming a hand piece structure with a cavity.

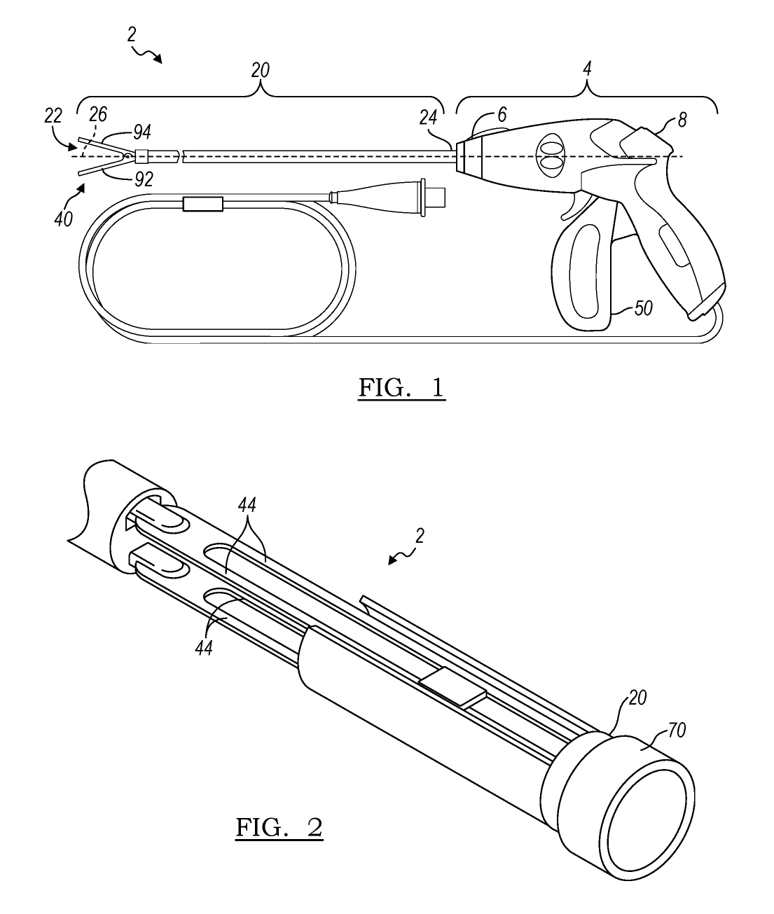

Turning now to FIG. 1, a side view of the forceps 2 is shown. The forceps 2 include a handpiece 4 having a distal end 6 and a proximal end 8. The handpiece 4 also includes at least one operable mechanism 50. A tubular member 20 has a proximal end 24 that is connected to the distal end 6 of the handpiece 4. The tubular member 20 includes a distal end 22 that includes jaws 40 extending therefrom. The jaws 40 have members 92 and 94 that open and close when the tubular member 20 is moved forward along the longitudinal axis 26 of the tubular member into contact with the members 92 and 94 or the jaws 40 are moved backwards along the longitudinal axis 26 into contact with the tubular member 20.

Referring further to FIGS. 2, 6 and 7A, a camming shaft 70 is located on the forceps 2 with the jaws 40 extending therefrom. The members 92 and 94 are biased by the camming shaft 70 so that the jaws 40 are opened and closed. A pair of slots 96 and 98 extend through the members 92 and 94, respectively. The member 92 includes a first compression surface 100 on a lateral portion of the member 92, that is on both sides of the slot 96, a second compression surface 104 on a medial portion on both sides of the slot 96, and a shearing surface 108 arranged orthogonally between the compression surfaces 100 and 104 on both sides of the slot 96. The member 94 includes a first compression surface 102 on a lateral portion of the member 94 on both sides of the slot 98, a second compression surface 106 on a medial portion on both sides of the slot 98, and a shearing surface 110 arranged orthogonally between the compression surfaces 102 and 106 on both sides of the slot 98.

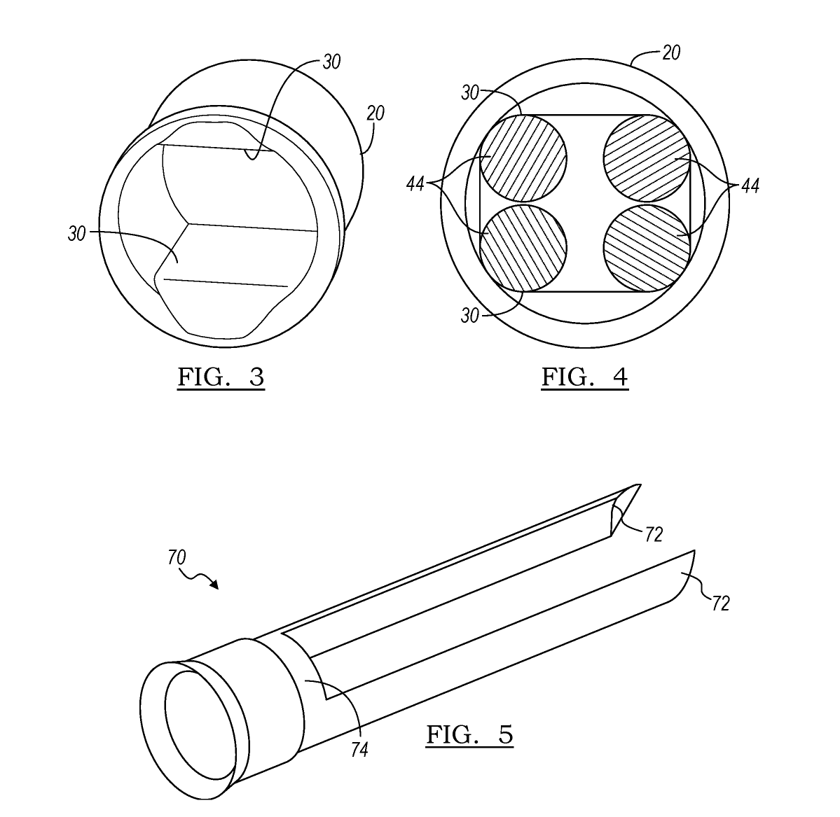

FIG. 3 illustrates the end of the tubular member 20 or a camming shaft showing a pair of internal flat portions 30 along the top surfaces and the bottom surfaces. FIG. 4 illustrates a cross-sectional view of a tubular member 20. The internal flat portions 30 include at least a portion that has a complementary shape to that of the legs of the jaws 44. Accordingly, as the tubular member 20 or the legs 44 axially move, the internal flat portions 30 control the orientation and movement of the jaws.

FIG. 5 illustrates a perspective view of one example of a camming shaft 70 that is inserted into the tubular member 20. The camming shaft 70 includes a molded flare 74 with a pair of protrusions 72 extending therefrom.

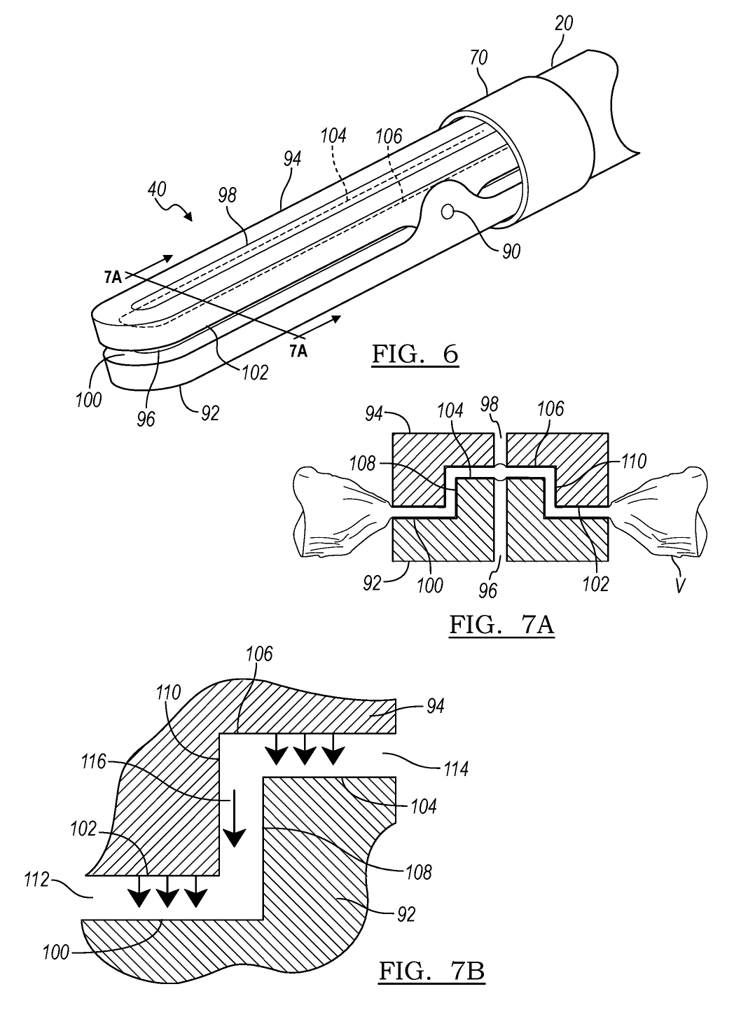

FIG. 6 illustrates the jaws 40 including a pin 90 located between the jaws. The pin 90 holds the jaw members 92 and 94 together and provide a pivot point for the jaw members 92 and 94 such that the members 92 and 94 close when the shaft 20 when the tubular member is slid over the opposing members 92 and 94.

Turning back to FIG. 7A, the jaw members 92 and 94 are shown clamping and sealing a vessel, V. The jaw members 92 and 94, as shown in FIG. 7B, form a combined compression zones 112 and 114 and a stretching and shearing zone 116 on the vessel, V, when the jaw members 92 and 94 are clamped together. One jaw member can move while the other is kept stationary, or both jaw members 92 and 94 can be moved together. In various arrangements, the gap between the upper and lower jaw members 92 and 94 in the compression zones 112 and 114 is between about 0.006 inch and 0.012 inch when the jaw members 92 and 94 are fully closed. The gap between the upper and lower jaw members in the shearing zone 116 is between about 0.003 inch and 0.006 inch when the jaw members are partially closed or fully closed.

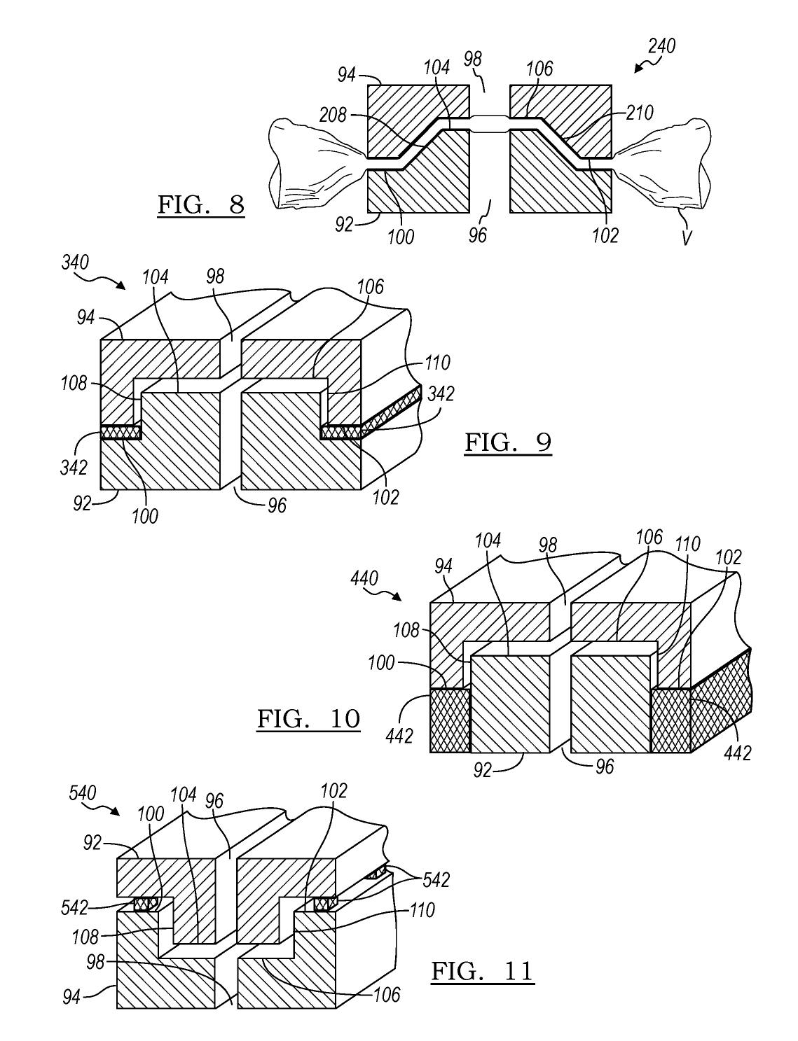

Turning now to FIG. 8, there is shown an alternative set of jaws 240 in accordance with the principles of the present invention. Some of the features of the jaws 240 are the same as those of the jaws 40 and are therefore identified by the same reference numbers. The jaws 240, however, has angled shearing surfaces rather than orthogonal shearing surfaces. Specifically, the jaw member 92 includes an angled shearing surface 208 between the compression surfaces 100 and 104 on both sides of the slot 96, and the jaw member 94 includes an angled shearing surface 210 between the compression surfaces 102 and 106 on both sides of the slot 98.

FIG. 9 illustrates yet another alternative set of jaws 340 in accordance with the principles of the present invention. The features of the jaw that are similar to the features of the jaws 40 are identified by the same reference numbers. The jaws 340 include two elongated insulation stop members 342 on both sides of the slots 96 and 98. The stop members 342 can be attached to either the compression surface 100 of the jaw member 92 or the compression surface 102 of the jaw member 94. The elongated insulation stop members 342 help control the gap between the jaw members 92 and 94 for vessel sealing. Further, the elongated insulation stop members 342 reduce thermal spread because the coagulation of the vessel, V, does not happen on the side edges of the jaw members 92 and 94.

FIG. 10 illustrates yet another alternative set of jaws 440 with elongated insulation stop members 442 located on both sides of the slots 96 and 98. The elongated insulation stop members 442 extend through the thickness of the jaw member 92 at the lateral portions of the jaw member 92. Alternatively, the elongated insulation stop members 442 can extend through the thickness of the jaw member 94. The benefits of the elongated insulation stop members 442 are similar to those of the elongated insulation stop members 342 described above.

FIG. 11 illustrates yet another set of jaws 540 which includes a set of gripping members 542. The members 542 are spaced apart along the length of the compression surface 100 of the jaw member 92 on both sides of the slot 96 or along the compression surface 106 of the jaw member 94 on both sides of the slot 98. The members 542 can be either conductive or non-conductive. These gripping members 542 apply tension to tissue across the jaw surfaces while holding the tissue in place. As such, the tissue is stretched into the jaw members 92 and 94 as they are closed together. In some arrangements, the gripping members 542 retract, for example, on pins on springs when the jaw members 92 and 94 are closed together. In other arrangements, the gripping members 542 are coincident with recesses in opposite jaw member, which allows the jaw members to close. In various other arrangements, the gripping members 542 are made of a compressible material, such as, for example, silicone rubber, that compresses under the force generated when the jaw members 92 and 94 are closed together. In any of the aforementioned arrangements, a further benefit of the gripping members 542 is that they can allow retraction or compression of the gripping members 542 until a gap, for example, between about 0.003 inch and 0.006 inch is achieved between the jaw members 92 and 94 to prevent inadvertent shorting of the jaw members 92 and 94 when they are configured as two electrodes energized by an electrical energy source.

Note that any of the members 342, 442, or 542 described above can be used in the jaws 240. Further note any of the aforementioned jaws enable stretching and thinning the vessel tissue by stretching, compressing and shearing the tissue before the jaws are energized to coagulate the tissue. In various implementations, shearing induces thinning of the vessel tissue and a state of increased tensile stresses in the tissue, that is, the shearing action stretches the tissue. In certain implementations, shearing increases the tensile stresses in the tissue to rupture or cut the tissue, that is, the shearing action severs the vessel.

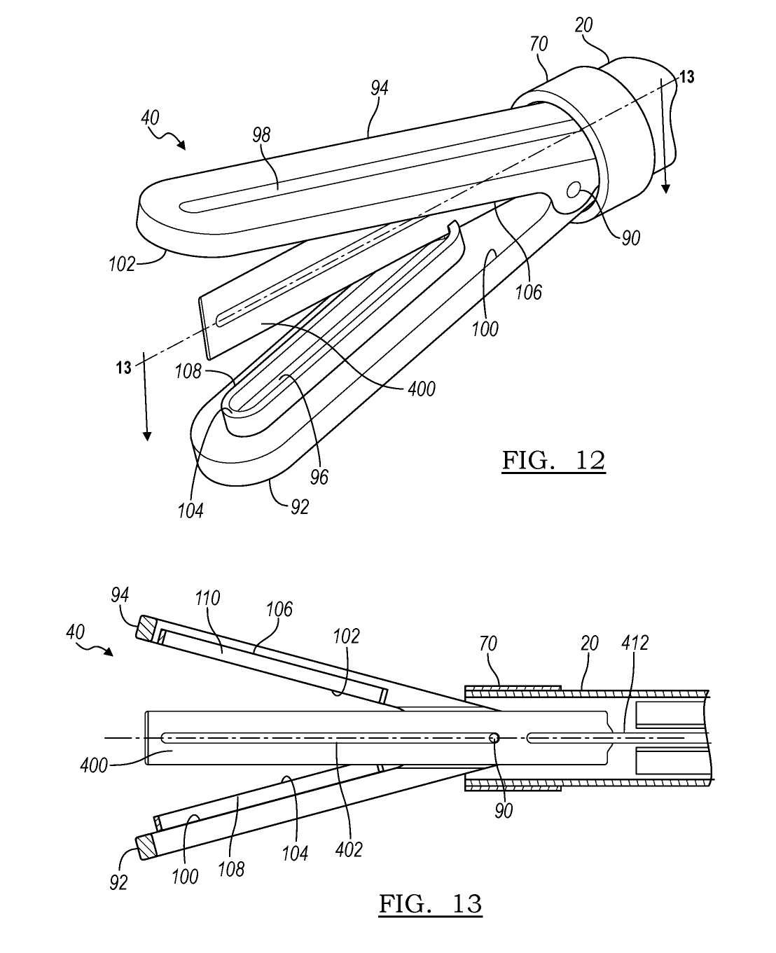

Any of the jaw arrangements 40, 240, 340, 440 and 540 described previously can include a cutting blade. For example, as shown in FIGS. 12 and 13, the jaws 40 are shown with a blade 400. The blade 400 includes a slot 402 that engages with the pin 90 to allow the blade 400 to reciprocate along the pin 90. The blade 400 is connected to a blade shaft 412. Hence, axial movement of the blade shaft 412 results in reciprocating axial movement of the blade 400 along the slots 96 and 98 of the jaw members 92 and 94 to cut tissue clamped between the jaw members 92 and 94. A similar blade arrangement can be implemented in the jaws 240, 340, 440 and 540.

The description of the invention is merely exemplary in nature and variations that do not depart from the gist of the invention are intended to be within the scope of the invention. Such variations are not to be regarded as a departure from the spirit and scope of the invention.

What is claimed is:

1. An end effector assembly of a forceps comprising:
a first jaw member having an electrically conductive tissue sealing surface configured to connect to a source of electrosurgical energy;
a second jaw member having an electrically conductive tissue sealing surface configured to connect to the source of electrosurgical energy,
wherein the first and the second jaw members are disposed in space opposition relation relative to one another, and at least one of the jaw members movable relative to the other between a first, open position and a second, closed position for the jaw members to grasp tissue therebetween,
wherein the tissue sealing surfaces of the first and the second jaw members are configured to form complementary stepped portions along an axis perpendicular to the longitudinal axis of the end effector assembly, the complementary stepped portions comprising a medial portion and a lateral portion on each of the first and second jaw, and
wherein one or both of the lateral surfaces has nonconductive stops.

2. The end effector assembly of claim 1 wherein the sealing surface of the first jaw member includes a first compression surface along the medial portion, a second compression surface along the lateral portion, and a shearing surface between the first compression surface and the second compression surface.

3. The end effector assembly of claim 2 wherein the sealing surface of the second jaw member includes a first compression surface along the medial portion, a second compression surface along the lateral portion, and a shearing surface between the first compression surface and the second compression surface.

4. The end effector assembly of claim 3 wherein the shearing surface of each of the jaw members is arranged orthogonally to the first compression surface and the second compression surface of the respective jaw member.

5. The end effector assembly of claim 3 wherein the shearing surface of each of the jaw members is arranged non-orthogonally to the first compression surface and the second compression surface of the respective jaw member.

6. The end effector assembly of claim 2 wherein the non-conductive stop is a gripping member positioned along the outermost compression surface of at least one of the jaw members, the non-conductive stop preventing inadvertent shorting between the jaw members.

7. The end effector assembly of claim 1 wherein the source generates electrosurgical energy to coagulate tissue grasped between the first jaw member and the second jaw member.

8. A forceps comprising:
an effector assembly comprising:
a first jaw member having an electrically conductive tissue sealing surface configured to connect to a source of electrosurgical energy;
a second jaw member having an electrically conductive tissue sealing surface configured to connect to the source of electrosurgical energy,
wherein the first and the second jaw members are disposed in space opposition relation relative to one another, and at least one of the jaw members movable relative to the other between a first, open position and a second, closed position for the jaw members to grasp tissue therebetween,
wherein the tissue sealing surfaces of the first and the second jaw members are configured to form complementary stepped portions along an axis perpendicular to the longitudinal axis of the end effector assembly, the complementary stepped portions comprising a medial portion and a lateral portion on each of the first and second jaw, and
wherein one or both of the lateral surfaces has nonconductive stops.

9. The forceps of claim 8 wherein the sealing surface of the first jaw member includes a first compression surface along the medial portion, a second compression surface along the lateral portion, and a shearing surface between the first compression surface and the second compression surface.

10. The forceps of claim 9 wherein the sealing surface of the second jaw member includes a first compression surface along the medial portion, a second compression surface along the lateral portion, and a shearing surface between the first compression surface and the second compression surface.

11. The forceps of claim 10 wherein the shearing surface of each of the jaw members is arranged orthogonally to the first compression surface and the second compression surface of the respective jaw member.

12. The forceps of claim 10 wherein the shearing surface of each of the jaw members is arranged non-orthogonally to the first compression surface and the second compression surface of the respective jaw member.

13. The forceps of claim 10 wherein the non-conductive stop is a gripping member positioned along the outermost compression surface of at least one of the jaw members, the non-conductive stop preventing inadvertent shorting between the jaw members.

14. The forceps of claim 8 wherein the source generates electrosurgical energy to coagulate tissue grasped between the first jaw member and the second jaw member.

15. The forceps of claim 8 wherein the tissue is gripped to provide tension, the forceps further including a reciprocating blade that cuts the tissue.

16. A method of using forceps, the method comprising:
opening a first jaw member and a second jaw member of the forceps, the first jaw member having an electrically conductive tissue sealing surface configured to connect to a source of electrosurgical energy and the second jaw member having an electrically conductive tissue sealing surface configured to connect to the source of electrosurgical energy, the first and the second jaw members being disposed in space opposition relation relative to one another, the tissue sealing surfaces of the first and the second jaw members being configured to form complementary stepped portions along an axis perpendicular to the longitudinal axis of the end effector assembly, the complementary stepped portions comprising a medial portion and a lateral portion on each of the first and second jaw, one or both of the lateral surfaces having nonconductive stops;
closing the jaw members to grasp tissue therebetween; and
pressing the jaw members together to cut tissue.

17. The method of claim 16 wherein the sealing surface of the first jaw member includes a first compression surface along the medial portion, a second compression surface along the lateral portion, and a shearing surface between the first compression surface and the second compression surface.

18. The method of claim 17 wherein the sealing surface of the second jaw member includes a first compression surface along the medial portion, a second compression surface along the lateral portion, and a shearing surface between the first compression surface and the second compression surface.

19. The method of claim 18 wherein the shearing surface of each of the jaw members is arranged orthogonally to the first compression surface and the second compression surface of the respective jaw member.

20. The method of claim 18 wherein the shearing surface of each of the jaw members is arranged non-orthogonally to the first compression surface and the second compression surface of the respective jaw member.

21. The method of claim 16 wherein the non-conductive stop is a gripping member positioned along the outermost compression surface of at least one of the jaw members, the non-conductive stop preventing inadvertent shorting between the jaw members.

22. The method of claim 16 further comprising generating electrosurgical energy to coagulate tissue grasped between the first jaw member and the second jaw member.

23. The method of claim 16 wherein the tissue is gripped to provide tension and the tissue is cut by a reciprocating blade.
